(19) United States
(12) Patent Application Publication
(10) Pub. No.: US 2001/0049357 A1
(43) Pub. Date: Dec. 6, 2001

(54) Use of Hsp27 as an anti-inflammatory agent

(76) Inventors: Asit K. De, Worcester, MA (US); Carol L. Miller-Graziano, Holden, MA (US)

Correspondence Address: GARY L. CREASON, Fish & Richardson P.C., 225 Franklin Street, Boston, MA, 02110-2804, US

(21) Appl. No.: 09/729,519

(22) Filed: Dec. 4, 2000

Related U.S. Application Data
(60) Provisional application No. 60/168,834, filed on Dec. 3, 1999.

(51) Int. Cl. A61K 38/16; C12N 5/08

(52) U.S. Cl. 514/12; 435/372

(57) ABSTRACT

A method of inhibiting an inflammatory response in a mammal, e.g., a human patient, is disclosed. The method includes administering a therapeutically effective amount of heat shock protein 27 (Hsp 27). The invention also includes a method of inducing in a mammal production of IL-10 and IL-12 by administering an effective amount of Hsp 27. Also disclosed is a method of using Hsp27 to promote dendritic cell maturation in vitro.

### STATEMENT AS TO FEDERALLY-SPONSORED RESEARCH

Work on the invention was funded in part by the federal government (NIH 2RO 1 GM 36214-13). Therefore, the government may have certain rights in the invention.

### BACKGROUND OF THE INVENTION

Systemic inflammatory responses, as well as exaggerated local inflammatory cytokine production, have been implicated in mediating multiple organ failure and rheumatoid arthritis (1-2). During shock inflammatory stress, heat shock proteins (Hsp), which are stress response proteins found in all species, are upregulated (3-5). These Hsp play a role in protecting cells during stress and inflammatory responses (3-6). The large Hsp have also been suggested as danger signals that first activate monokine production, then stimulate and/or regulate the magnitude of the immune response (7-8). Some members of the large Hsp family (Hsp 60, 70) are effective in the prevention and treatment of experimental arthritis (6, 9, 10). The development of Hsp 60-specific Th2 cells producing IL-4 and IL-10 corresponds to the remission of rheumatoid arthritis in patients and these T cells suppress patient TNFα production (5, 9). Immunization of mice with Hsp 65 protects against pristane induced arthritis by inducing IL-10 and IL-4 producing CD4 T cells (12). IL-4 and IL-10 are potent downregulators of monocyte production of proinflammatory mediators, such as TNFα, IL-8, IL-1 and PGE2 (13-15). Human Hsp 60 has been shown to induce TNFα in a human monocyte cell line and TNFα, as well as IL-15 and IL-1 2, in murine bone marrow derived macrophage (BMDM).

Hsp 27, a member of the small Hsp family, has been investigated for its role as a circulating protein marker of increased malignancy in breast cancer (16). Hsp 27 downregulates reactive oxygen intermediates (ROI) production, thereby protecting from TNFα mediated apoptosis (17). Circulating Hsp 27 is present in the serum of cancer patients and induces in vivo Hsp 27 antibody production, suggesting that Hsp 27 can stimulate as an exogenous protein (23, 24). Phosphorylated Hsp 27 also has been associated with cell membranes of lamellipodia in migrating cells, suggesting a possible Hsp 27 surface expression (25). Administration of IL-10 has been shown to suppress lethal endotoxemia and reduce serum TNFα levels (26). Because of its anti-inflammatory properties, IL-10 has been suggested as a possible therapeutic agent for inflammatory conditions, such as rheumatoid arthritis and inflammatory bowel disease (26). However, IL-10 also has immunosuppressive effects.

### SUMMARY OF THE INVENTION

It has been discovered that exogenous Hsp 27 induces production of IL-10 (an anti-inflammatory cytokine) and IL-12 (an immunostimulatory cytokine) in human monocytes (MØ). In addition, it has been discovered that Hsp 27 induction ofIL-10 and IL-12 involves certain MAPKinase pathways during Hsp 27 induced MØ IL-10 production, and that Hsp 27 induces high levels of MØ IL-10 while concomitantly stimulating only minimal levels of TNFα. Hsp27 induction of IL-10 appears to depend on activation ofthe p38 MAPKinase pathway.

Based on these discoveries, the invention provides a method of inhibiting an inflammatory response in a mammal, e.g., a human patient. The method includes administering to the mammal a therapeutically effective amount of Hsp 27. The invention also includes a method of inducing in a mammal production of IL-10, IL-12, or both simultaneously, by administering to the mammal an effective amount of Hsp 27.

For inhibiting an inflammatory response, or for inducing production of IL-10, IL-12, or both, the therapeutically effective amount preferably is from 1 μg/kg to 160 μg/kg. In some embodiments of the invention, the therapeutically effective amount is 2 μg/kg to 80 μg/kg, e.g., from 4 μg/kg to 40 μg/kg.

The invention also provides an anti-inflammatory composition comprising an effective amount of Hsp 27 and a pharmaceutically acceptable carrier.

The invention also provides a method of promoting dendritic cell maturation in vitro. The method includes the steps of: isolating monocytes from blood without triggering activation of the monocytes; culturing the monocytes in vitro; inducing conversion of the monocytes into immature dendritic cells; and contacting the dendritic cells with an effective amount of Hsp27 for an effective length of time, thereby promoting maturation of the dendritic cells. Inducing conversion of the monocytes into immature dendritic cells can be achieved, for example, by culturing the monocytes in a medium containing interleukin-4 (IL-4) and granulocyte macrophage colony stimulating factor (GMCSF) for an effective conversion time. An effective conversion time preferably is from 2 to 5 days, and often is 3 or 4 days. Preferably, the effective amount of Hsp27 is 0.1 μg/ml to 500 μg/ml, and more preferably, it is 1 μg/ml to 100 μg/ml, e.g., 5 μg/ml to 50 μg/ml.

The invention also provides a method of enhancing an immune system response in a human patient. The method includes: collecting a sample of blood from the patient; isolating monocytes from the blood without triggering activation of the monocytes; culturing the monocytes ex vivo; inducing conversion of the monocytes into immature dendritic cells; promoting maturation of the dendritic cells by contacting the dendritic cells with an effective amount of Hsp27 for an effective length of time; and reintroducing the dendritic cells into the patient. In some embodiments of the invention, the method further includes the step of contacting the dendritic cells with an antigen after promoting maturation of the dendritic cells, and before reintroducing the dendritic cells into the patient. The antigen can be, for example, a human tumor antigen, a bacterial antigen, and a viral antigen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed descriptions, and from the claims.

### BRIEF DESCRIPTION OF THE DRAWINGS

FIG. 1A is a bar graph that depicts the results of experiments in which human MØ were cultured (1×10⁶ cells/ml) for 16-18 hrs in the presence or absence of muramyl dipeptide (MDP) (20 μg/ml) plus Staphylococcal enterotoxin B (SEB) (0.5 μg/ml) or recombinant human Hsp 27 (2 μg/ml). IL-10 levels in the culture supernates were tested by ELISA. Data are expressed as means ± SEM. Representative of seven experiments. *p=0.0001 and **p=0.0009 as compared to only adherence stimulated MØ IL-10 levels.

FIG. 1B is a graph summarizing the results of experiments in which human MØ were cultured as in FIG. 1A in the presence of different concentrations of Hsp 27 and the culture supernates tested for IL-10 levels. Representative of three experiments.

FIG. 2A is a histogram summarizing data from experiments showing that Hsp 27 induces MØ IL-10 production is not due to endotoxin contamination in the recombinant Hsp 27 preparation. Human MØ were cultured (1×10⁶ cells/ml) for 16-18 hrs in the presence of Hsp 27 (2 μg/ml) alone or in combination with polymyxin B (200 U/ml) and then tested for IL-10 levels in the culture supernates. Data are expressed as means ± SEM, and are epresentative of five experiments.

FIG. 2B is a histogram summarizing data from experiments in which Hsp 27 was incubated with anti-Hsp 27 (20 μg/ml, final concentration) for 3 hrs before its addition to the MØ culture. Representative of three experiments. *p=0.016 as compared to only Hsp 27 induced MØ IL-10 levels.

FIG. 3A is a histogram summarizing data from experiments showing Hsp 27 induction of TNFα in human MØ. Cells were cultured (1×10⁶ cells/ml) for 16-18 hrs in the presence or absence of MDP (20 μg/ml) plus SEB (0.5 μg/ml) or Hsp 27 (2 μg/ml). TNFα levels in the culture supernatants were tested by ELISA. Data are expressed as mean ± SEM. Representative of seven experiments. *p=0.009 and **p=0.0003 as compared to only adherence stimulated MØ TNFα levels.

FIG. 3B is a histogram summarizing experimental data showing the results of culturing human MØ as in FIG. 3A in the presence of Hsp 27 alone or in combination with anti-TNFα antibody (10 μg/ml) and then testing for IL-10 levels in the culture supernatants. Representative of five experiments. ***p=0.03 as compared to Hsp 27 induced IL-10 levels.

FIG. 4 is a photograph of a series of gels showing experimental activation (phosphorylation) of different MAPKinase pathways in human monocytes by Hsp 27. 1.5×10⁶ MØ were cultured for 2 hrs in serum-free medium, followed by stimulation with Hsp 27 (2 μg/ml) for different time periods (1-180 mins). Cells were lysed as detailed in the Methods. Equal amounts of the postnuclear lysates were immunoblotted (SDS-12% PAGE followed by transfer to nitrocellulose membrane) with anti-phospho-p38 MAPK antibody. The same membranes were used for detection of other MAPK (both phosphorylated and total) by sequential stripping of the membranes, followed by reprobing the blots with respective antibody. Representative of three experiments.

FIG. 5 is a histogram summarizing results of experiments showing that Hsp 27 induces MAPKAPKinase-2 activity in human monocytes. 1.5×10⁶ MØ were cultured in serum-free medium for 2 hrs and then stimulated with MDP (20 μg/ml)+SEB (0.5 μg/ml), Hsp 27 (2 μg/ml) or UV (as positive control) for 30 mins. Cells were then lysed and the postnuclear lysates were used for assessment of MAPKAPK-2 activity by immunoprecipitation of the enzyme by anti-MAPKAPK-2 antibody, followed by in vitro kinase assay using Hsp 27 peptide sequence (KKLNRTSVA) as the substrate. Incorporation of [α-³²P] ATP into the substrate were assessed by scintillation counting and expressed as CPM. Representative of three experiments. *p=0.04 and **p=0.03 as compared to only adherence stimulated MØ MAPKAPK-2 activity.

FIG. 6 is a histogram summarizing results of experiments demonstrating that SB203580, but not PD98059 inhibits Hsp 27-induced MØ IL-10 production. MØ (1×10⁶ cells/ml) were treated with SB203580 (10 μM) or PD98059 (10 μM) for 2 hrs before addition of Hsp 27 (2 μg/ml) to the MØ culture. MØ were then cultured for 16-18 hrs and tested for IL-10 or TNFα levels in the culture supemates. Data are expressed as mean ± SEM. Representative of seven experiments for IL-10 production and of five experiments for TNFα production. *p=0.002 as compared to Hsp 27 induced IL-10 levels, #p=0.002 as compared to only adherence stimulated IL-10 levels, **p=0.04 as compared to Hsp 27 induced TNFα levels.

FIGS. 7A-7C are histograms summarizing data on induction of IL-10 by Hsp27 in human MØ, as compared to other stimuli. FIG. 7A shows mean IL-10 level in supernates from MØ cultures stimulated by adherence alone, a combination of muramyl dipeptide (MDP) and SEB, or Hsp27. FIG. 7B shows mean IL-10 level in supernates from MØ cultures stimulated by adherence alone, Zymosan, or Hsp27. FIG. 7C shows mean IL-10 level in supernates from MØ cultures stimulated by adherence alone, Hsp27, or Hsp27 plus αHsp27.

FIGS. 8A and 8B are histograms summarizing data on induction of IL-12 by Hsp27 in human MØ, as compared to other stimuli. FIG. 8A shows mean IL-12 level in supernates from MØ cultures stimulated by adherence alone, a combination of MDP and SEB, or Hsp27. FIG. 8B shows mean IL-12 level in supernates from MØ cultures stimulated by adherence alone, Zymosan, or Hsp27.

FIG. 9 is a histogram summarizing data on induction of TNFα by Hsp27 in human MØ, as compared to other stimuli. FIG. 9 shows mean MØ TNFα in supernates from MØ cultures stimulated by adherence alone, a combination of MDP and SEB, Hsp27, or Zymosan.

FIGS. 10A-10C are histograms summarizing data on restorationi of trauma patients' monocyte IL-10 and IL-12 levels after stimulation with Hsp27, expressed as median percentage of normal IL-10 level. FIG. 10A shows IL-10 levels in MØ treated with MDP plus SEB, or Hsp27. FIG. 10B shows IL-12 levels in MØ treated with MDP plus SEB, or Hsp27. FIG. 10C shows IL-12 levels in MØ treated with zymosan or Hsp27.

### DETAILED DESCRIPTION

Human Hsp 27 has unique potential as in vivo therapy for pathologic inflammatory conditions for several reasons. Hsp27 is a natural, endogenous protein, so it is predicted to have few side effects. Hsp27 is a potent inducer of IL-10, a known anti-inflammatory stimulus. At the same time, Hsp 27 is a potent inducer or MØ Il-12 production. Simultaneous induction of both IL-12 and IL-10 results in the immunodepressive effects of IL-10 on T lymphocytes being minimized by the pressure of IL-12, while the anti-flammatory effect of IL-10 is maintained.

Hsp27 analogs include mutant forms of the native or wild-type Hsp27 that have the same or similar biological activity as wild-type Hsp27. Such mutant forms can include conservative amino acid substitutions of one or more, e.g., 1-20, naturally occurring amino acids in wild-type Hsp27. Conservative amino acid substitutions can be made using conventional techniques. Other types of Hsp27 analogs, e.g., Hsp27 fusion proteins or truncated fragments of wild-type Hsp27, can be obtained by conventional methods.

Part of the MØ IL-10 levels induced by Hsp 27 stimulation are due to its prior induction of TNFα, a known enhancer of IL-10 in MØ. However, Hsp 27 directly induces high levels of MØ IL- 10 while concomitantly stimulating only minimal levels of TNFα. IL-10 induction by Hsp27 depends on activation of the p38 MAPKinase pathway. Both IL-10 and IL-12 are significantly depressed in trauma patients who have high multiple organ dysfunction syndrome (MODS) scores. Hsp 27 advantageously induces IL-10 and IL-12 in monocytes from immunosuppressed patients (Example 7).

Although other known stimulants of monocytes induce simultaneously equivalent amounts of pro- and anti-inflammatory cytokines, hsp 27 preferentially induces large quantities of anti-inflammatory cytokines (IL-10). This makes Hsp 27 particularly suitable for anti-inflammatory therapy. Hsp 27 therapy offers advantages over administration of IL-10, because Hsp 27 induces IL-12, an immunostimulatory cytokine that activates T lymphocytes. This counterbalances the immunosuppressive effects of IL-10. Consequently, in vivo treatment with Hsp 27 is potentially anti-inflammatory but not immunosuppressive. An additional advantage to the invention is that Hsp 27 is a normal human protein and therefore will not be antigenic.

Effective Dose

A therapeutically effective dose is an amount of Hsp27 sufficient to achieve amelioration of symptoms of an inflammatory response or disorder, e.g., rheumatoid arthritis or inflammatory bowel disease. Toxicity and therapeutic efficacy of therapeutic compounds (i.e., Hsp 27 and Hsp 27 analogs) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

Data obtained from cell culture assays and animal studies can be used in designing a dosage range for use in humans. Dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography. An example of a dose is from 1-200 mg/kg body weight in a human. Another example is from 10-50 mg/kg body weight in a human.

Promoting Dendritic Cell Maturation

In some embodiments of the invention, a cell population, e.g., a population highly enriched for MØ, is isolated from blood collected from a patient undergoing therapy according to the invention. The isolated cells placed into culture, treated with Hsp27 and other agents, and re-introduced into the patient.

Analogous treatment can be carried out using laboratory animals, e.g., in pre-clinical studies. When non-human animals are treated according to the invention, the Hsp27 analog for the laboratory animal substituted for Hsp27. For example, the murine analog of Hsp27 is Hsp25.

The amount of blood collected for MØ isolation can vary according to the age and condition of the patient. Preferably, 10 to 100 ml, e.g., 25 to 50 ml, of blood is collected, and MØ are isolated according to conventional methods. Methods for isolating MØ are known in the art and can be employed without undue experimentation. For example, MØ can be isolated by negative selection, without causing MØ activation.

In a preferred method for obtaining dendritic cells in vitro, isolated MØ are are first stimulated to undergo conversion (differentiation) into immature dendritic cells, and then stimulated to mature into fully active or competent dendritic cells. Conversion can be stimulated or promoted in vitro by any effective treatment. For example, conversion can be promoted by treating the MØ with an effective amount of IL-4 and an effective amount of GM-CSF, in accordance with conventional techniques. Typically, the IL-4 and GM-CSF treatment is for approximately 3-4 days. One indication of conversion is expression of CD1a. For guidance concerning in vitro conversion of MØ to immature dendritic cells, see, e.g., Chapius et al., 1997, Eur. J. Immunol. 27:431-441.

In methods of the invention, maturation of immature dendritic cells is stimulated or promoted by treating the immature dendritic cells with an effective amount of Hsp27.

Preferably, the MØ are not brought into contact with Hsp27 before their conversion into immature dendritic cells, because Hsp27 inhibits the conversion. After conversion, however, Hsp27 acts as a potent promoter of maturation by the immature dendritic cells. Maturation time is advantageously reduced in immature dendritic cell populations treated with exogenous Hsp27, as compared to immature dendritic cell populations not treated with exogenous Hsp27. Dendritic cell maturation is detectable as early as 24 hours after initiation of Hsp27 treatment. Preferably, however, Hsp27-induced maturation is allowed to proceed for 48 to 72 hours. One useful indication of dendritic cell maturation is expression of CD83.

Methods for re-introducing dendritic cells into patients are known in the art and can be employed in the practice of the present invention. See, e.g., Timmerman et al., 1999, Annual Review of Medicine 50:507-529.

Optionally, the mature dendritic cells can be exposed to (“loaded”) with one or more antigens, prior to being re-introduced into the patient. Antigen loading can be used to enhance the patient's immune response to particular antigens, e.g., a tumor antigen or an antigen found on a particular infectious agent. Methods for antigen loading are known in the art. See, e.g., Schuler et al., 1997, Int. Arch. Allergy and Immunol. 112:317-322.

Formulations And Use

Pharmaceutical compositions for use in accordance with the present invention can be formulated in conventional manner using one or more physiologically acceptable carriers or excipients. Thus, the compounds and their physiologically acceptable salts and solvates can be formulated for parental administration or administration by inhalation or insufflation (through the mouth or the nose), or rectal administration.

For administration by inhalation, the compounds for use according to the present invention are conveniently delivered in the form of an aerosol spray from pressurized packs or a nebulizer, using a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Long acting formulations, e.g., encapsulated microspheres can be administered by injection or implantation, which can be subcutaneous or intramuscular. The administered compounds can be formulated with suitable polymeric or hydrophobic materials (for example as an emulsion in an acceptable oil) or ion exchange resins, or as sparingly soluble derivatives, for example, as a sparingly soluble salt.

The following examples are provided to further illustrate the invention. The following examples are provided for illustrative purposes only, and are not to be construed as limiting the scope or content of the invention in any way.

### EXAMPLES

### Example 1.

### Materials and Methods

Reagents

Fetal bovine serum (FBS) was purchased from Sigma Chemical Co (St. Louis, Mo.). Culture media and other supplements were purchased from Irvine Scientific (Santa Ana, Calif.). Muramyl dipeptide (MDP) was provided by CIBA-GEIGY Limited (Basel, Switzerland). SEB was purchased from Sigma (St. Louis, Mo.) and polymyxin B was from Calbiochem Corp. (LaJolla, Calif.). The monoclonal antibodies, My4 (CD14)-FITC and IgG2b-FITC were purchased from Coulter Corp (Hialeah, Fla.). Recombinant human heat shock protein-27 (Hsp 27) was purchased from Stressgen Biotechnologies Corp. (Victoria, Canada). Polyclonal antibody against Hsp 27 was purchased from Santa Cruz Biotechnology, Inc. (Santa Cruz, Calif.), and monoclonal antibody against TNFα from Endogen, Inc. (Woburn, Mass.). SB203580 and PD98059 were purchased from Calbiochem Corp. Phosphoplus p38 MAPK, p44/42 (Erk 1/2) MAPK and SAPK/JNK kits were purchased from New England Biolabs, Inc. (Beverly, Mass.). MAPKAPKinase-2 IP-Kinase Assay kit was purchased from Upstate Biotechnology (Lake Placid, N.Y.). [³²p] and ECL reagents were purchased from NEN Life Science Products, Inc. (Boston, Mass.).

Separation of MØ and stimulations

Peripheral blood mononuclear cells (PBMC) were first isolated from venous blood of healthy human volunteers by Ficoll-Hypaque density centrifugation. MØ were separated from PBMC by selective adherence to microexudate-coated plastic surfaces as described (28). Adherent MØ (>95% purity, as checked by flow cytometric analysis) were collected by treatment with 10 mM EDTA, suspended in IMDM medium, supplemented with 10% FBS, 50 U/ml penicillin-G, 50 μg/ml streptomycin, 50 μg/ml gentamycin, 2.5 μg/ml fungizone, 4 mM L-glutamine, 1 mM sodium pyruvate, and 1% minimal essential medium non-essential amino acids. Endotoxin contamination was less than 12 pg/ml in the culture medium and FBS. Polymyxin B was added (20 U/ml) to all the washing and culture media to block the effect of any contaminating LPS. In some experiments, polymyxin B was used at a higher concentration (200 U/ml) in MØ culture. MØ were cultured (1×10⁶ cells/ml) for 16-18 hrs in the presence or absence of 20 μg/ml of muramyl dipeptide (MDP)+SEB (0.5 μg/ml) or human Hsp 27 (2 μg/ml). Culture supemates were harvested and stored at −80° C. until they were tested for IL-10 or TNFα. In selected experiments, Hsp 27 was first incubated with α-Hsp-27 polyclonal antibody (20 μg/ml) for 3 hrs before its addition to MØ culture or α-TNFα monoclonal antibody (10 μg/ml) was added, together with Hsp 27, to MØ culture. In some experiments, MØ were first treated with SB203580 (10 μM), or PD98059 (10 μM), or the DMSO control (solvent used for dissolving both the reagents) for 2 hrs before addition of Hsp 27 to the culture.

RNAse protection assay (RPA)

2×10⁶ monocytes were stimulated in the presence or absence of MDP (20 μg/ml)+SEB (0.5 μg/ml) or Hsp 27 (2 μg/ml) for 8-9 hrs. Total cytoplasmic RNA was isolated using Tri-reagent (Molecular Research Center, Inc., Cincinnati, Ohio), according to manufacturer's instructions. Antisense probes were labeled with ³²P-UTP (NEN Life Science Products, Inc.) using the Riboquant in vitro transcription labeling kit (Pharmingen, San Diego, Calif.), according to manufacturer's instructions. A cocktail of probes, Riboquant hCK-1 (Pharmingen, San Diego, Calif.), is used to facilitate the simultaneous quantification of several RNA species. The antisense probes generated using this probe set include the controls—GAPDH and L32 and the human cytokine IL-10 and some other human cytokines—IL-5, IL-4, IL-14, IL-15, IL-9, IL-2, IL-13, and IFNγ. The ribonuclease protection assays were performed using the Riboquant RPA kit (Pharmingen, San Diego, Calif.), according to manufacturer's instructions. In brief, molar excesses of labeled probes were incubated with RNA derived from cells in hybridization buffer supplied by the manufacturer for 16-48 hrs at 56° C. Hybridized samples were then digested with 5 U of RNAse A/T1 mixture for 45 mins at 30° C. Subsequent to digestion, the protected fragments were separated from digested probe by electrophoresis on an 8 molar urea 5% polyacrylamide TBE gel. The gels were then dried, exposed directly to film and developed. The band intensities were quantitated using the NIH image software. IL-10 mRNA levels were adjusted according to L32 and GAPDH levels (used as loading controls).

Immunoblot assay for activation (phosphorylation) of Erk 1/2, SAPK/JNK and p38 MAPK.

Monocytes (1.5×10⁶ cells) were cultured in serum free medium for 2 hrs and then stimulated with Hsp 27 (2 μg/ml) for different time periods (1 min to 3 hrs). Western blot analysis was performed, essentially as described previously (29). Briefly, cells were lysed using a buffer consisting of 1% Nonidet P-40, 50 mM HEPES (pH 7.2), 100 mM NaCl, 2 mM EDTA, 1 mM pyrophosphate, 2 mM Na₃VO₄, 10 mM NaF, 1 mM PMSF, 10 μg/ml leupeptin and 10 μg/ml aprotinin. Postnuclear supernates were harvested after centrifugation of the lysate for 15 min at 14,000 g at 4° C. Equal amounts of postnuclear lysates were boiled for 5 min in the presence of SDS sample buffer (reducing) and subjected to SDS-12% PAGE and then transferred to nitrocellulose membrane (Millipore Corp, Bedford, Mass.) in transfer buffer [25 mM Tris, 192 mM glycine, pH 8.3, 20% (V/V) methanol]. Membranes were first rinsed in TTBS (TBS with 0.1% Tween 20) and then blocked for 1 hr at room temperature in TTBS-5% W/V nonfat dry milk. The membrane was then incubated overnight at 4° C. with antiphospho-p38 MAPK antibody (rabbit polyclonal; New England Biolab) (1:1000 dilution in TTBS-1% BSA). Antibody-antigen complexes were detected with the aid of HRP-conjugated anti-rabbit secondary antibody (1:2000 dilution) (New England Biolab) followed by detection of the bands with ECL reagent (NEN Life Science Products). The same membranes were used for detection of several other proteins—such as Phospho-Erk 1/2 (p44/42), Phospho-SAPK (stress activated protein kinase)/JNK, total p38 MAPK, total Erk 1/2 and total SAPK/JNK by sequential stripping of antibodies, by incubation of the membrane for 30 min at 50° C. in a specific buffer (2% SDS, 100 mM 2-ME, 62.5 mM Tris-HCl, pH 6.7) and then reprobing the blot with respective antibody (all antibodies—New England Biolab) using the procedure as mentioned above for the assessment of phospho-p38 MAPK.

MAPKAPKinase-2 assay

Monocytes (1.5×10⁶) were cultured in serum-free medium for 2 hrs and then stimulated with MDP(20 μg/ml)+SEB(0.5 μg/ml), Hsp 27 (2 μg/ml) or UV (as positive control) for 30 min. Postnuclear lysates were prepared as described above. Protein (A+G) (20μl of beads/sample) (Santa Cruz Biotechnology, Inc.) was first washed twice with ice-cold PBS and then the MAPKAPKinase-2 assay was performed as described, using a specific kit (Upstate Biotechnology) (13). In brief, washed Protein (A+G) was incubated with anti-MAPKAPKinase-2 sheep polyclonal antibody for 1 hr at 4° C. In some experiments, Protein (A+G) was incubated with sheep IgG for the antibody control. Antibody-bound Protein (A+G) was then washed twice with ice-cold PBS, followed by incubation with the postnuclear lysate sample for 2 hrs at 4° C. in ice-cold RIPA buffer (50 mM Tris, pH 7.5, 1 mM EDTA, 1 mM EGTA, 1 mM Na₃VO₄, 0.1% 2-ME, 1% Triton X-100, 5 mM sodium pyrophosphate, 10 mM sodium glycerophosphate, 0.1 mM PMSF, 1 μg/ml aprotinin, 1 μg/ml leupeptin and 50 mM NaF) with thorough mixing. The Protein (A+G)-enzyme immune complex was washed once with ice-cold RIPA buffer containing 0.5 M NaCl and then twice with ice-cold RIPA buffer and once with kinase assay buffer (20 mM MOPS, pH 7.2, 25 mM β-glycerol phosphate, 5 mM EGTA, 1 mM Na₃VO₄, 1 mM dithiothreitol). The beads were resuspended in 10 μl of kinase assay buffer, followed by addition of 10 μl of 1 mM heat shock protein-27 peptide sequence KKLNRTSVA (used as substrate). Reactions were initiated by the addition of 10 μl of [γ-³²p] ATP (10 μCi/assay) diluted in magnesium/ATP cocktail (75 mM magnesium chloride and 500 mM ATP in kinase assay buffer). The reaction was allowed to proceed for 30 min at 30° C. before termination. This was achieved by spotting the assay mixture onto squares of p81 paper and then placing them in 0.75% ortho-phosphoric acid. The squares were washed three times in the acid and once in acetone before scintillation counting.

Flow cytometric analysis

Cell phenotype verification in our monocyte populations was carried out using anti-CD 14 monoclonal antibody. IgG 2b-FITC was used as the isotype control. Fluorescent measurements were done on the Coulter Epics XL flow cytometer. Briefly, 5×10⁵ cells were incubated with conjugated mAb or with the appropriate isotypic control for 1 hour at dilutions suggested by the manufacturer. Samples were washed twice with PBS and resuspended in 500 μl PBS for fluorescent analysis.

ELISA assay of IL-10 and TNFα

IL-10 and TNFα levels in the culture supernatants were determined by specific ELISA kit (Endogen, Inc.) according to the instructions of the manufacturer. The sensitivity of the assay was 5 pg/ml.

Statistical analysis

Results are expressed as mean ± SEM. Statistical significance was calculated by the Student's T test (paired) using the StatView program. Statistical significance was accepted for p<0.05.

### Example 2.

### Hsp 27 Induction of IL-10 in Human MØ

To investigate a novel anti-inflammatory role for Hsp 27 in inducing MØ IL-10 production, we treated human MØ with recombinant human Hsp 27 and assayed IL-10 levels in the culture supernates. A combination of SEB plus MDP was used as a positive control for induction of MØ IL-10. LPS is a more frequently used potent stimulant of MØ IL-10 production (30). However, other stimuli, such as MDP, also induce significant quantities of IL-10 in MØ/macrophage, even in the presence of polymyxin B (28, 30). Consequently, we used MDP+SEB as a control stimuli so that polymyxin B could be included in all media and monokine production induced exclusively by Hsp 27 could be distinguished from that induced by Hsp 27 and any possible endotoxin contamination in the recombinant Hsp 27 preparation.

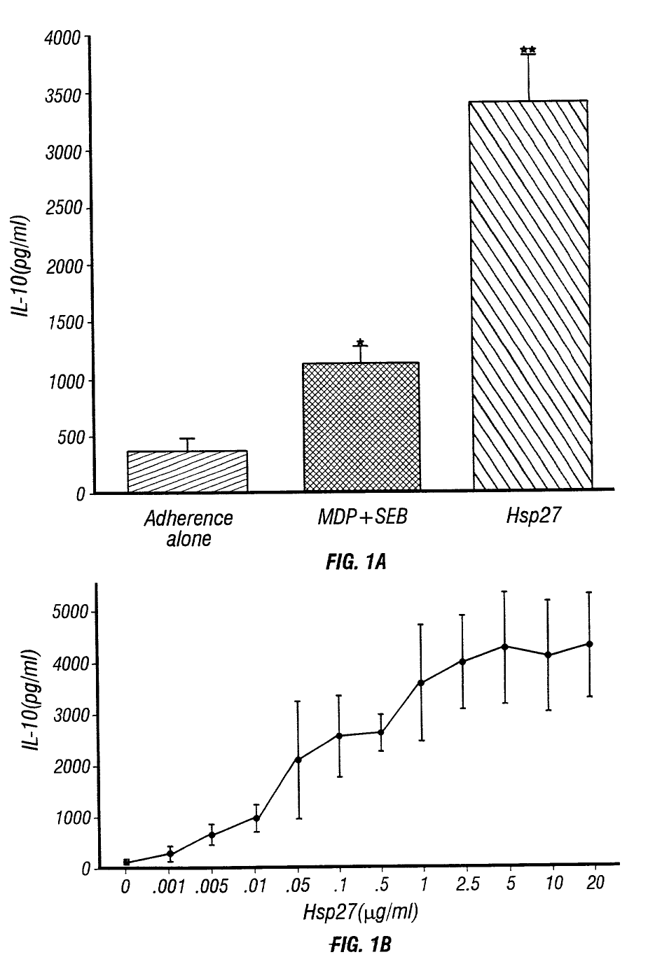

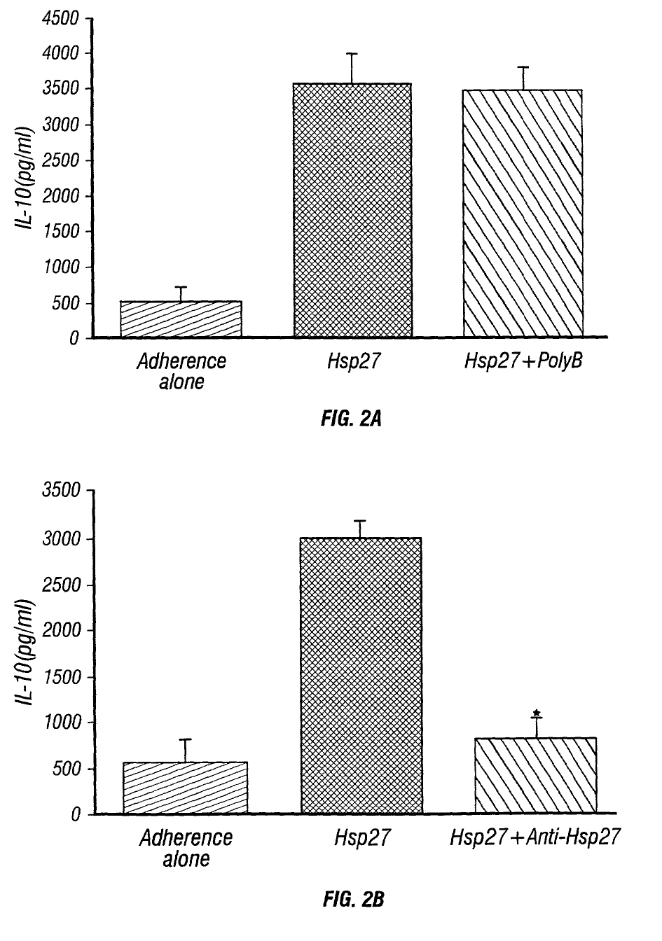

Hsp 27 induced significantly (p=0.0009) higher amounts of IL-10 as compared to adherence stimulated, untreated MØ or even SEB+MDP stimulated MØ (FIG. 1A). The Hsp 27 induction of IL-10 protein was maximal (about a 10 fold increase) at 16-18 hours and did not increase over an additional 48 hours culture. Although SEB+MDP induced MØ IL-10 levels continued to increase (still only to 3 fold increase over unstimulated) up to 40 hours in culture, SEB+MDP induced IL-10 levels never reached those induced by Hsp 27 at 18 hours Combination of Hsp 27 with SEB+MDP only minimally increased IL-10 induction over Hsp 27 alone (approximately 3400 to 3700 pg/ml), suggesting maximal IL-10 levels were induced by Hsp 27. Hsp 27 induced MØ IL-10 levels were approximately 10 fold higher than the untreated MØ IL-10 levels, whereas MDP+SEB induced MØ IL-10 levels were only about 3 fold higher than the untreated MØ IL-10 levels (FIG. 1A). Hsp 27 induced MØ IL- 10 production was dose-dependent, with 1-5 μg/ml being the optimum concentration (FIG. 1B). Although our culture media contained 20 U/ml of polymyxin B, it was still possible that the recombinant Hsp 27 was contaminated with high concentrations of endotoxin (LPS), which were not neutralized by the quantity of polymyxin B used in culture. Such LPS contamination might be responsible for augmented, interactive induction of MØ IL-10 by the Hsp 27 preparation. To test that possibility, we either added higher quantities of polymyxin B (200 U/ml) together with Hsp 27 in the MØ culture, or treated Hsp 27 with anti-Hsp 27 antibodies for 3 hrs before addition to the MØ culture. High concentrations of polymyxin B could not inhibit Hsp 27 induced MØ IL-10 production (FIG. 2A). On the other hand, anti-Hsp 27 treatment could abolish the potential of Hsp 27 for induction of MØ IL-10 production (FIG. 2B). These findings suggest that Hsp 27 itself induced MØ IL-10.

### Example 3.

### Hsp 27 Induces MØ IL-10 at the Level of mRNA

It was known that dherence alone can induce IL-10 in human MØ in the absence of any other stimulants (28). Consequently, we thought that Hsp 27 might be inducing MØ IL-10 protein levels by augmenting IL-10 protein translation of adherence stimulated, already transcribed IL-10 mRNA rather than by inducing increased additional transcription of the IL-10 gene. To explore this possibility, we assessed MØ IL-10 mRNA expression with the RNAse protection assays.

To demonstrate that Hsp 27 induces IL-10 mRNA in human monocytes, 2×10⁶ MØ were stimulated in the presence or absence of MDP (20 μg/ml)+SEB (0.5 μg/ml) or Hsp 27 (2 μg/ml) for 8-9 hrs and then total cytoplasmic RNA was isolated. Multiprobe RNAse protection assays were performed to measure the mRNA levels for IL-10 and also L32 and GAPDH (loading controls). Equivalent amounts of RNA were treated with ³²P-UTP-labeled Riboquant hck-1 probe cocktail and then digested with RNAse A/T₁ mixture. The protected fragments were then analyzed by electrophoresis on an 8 molar urea, 5% polyacrylamide TBE gel followed by drying of the gel and autoradiography. The gel was exposed for 6 hours to assay the IL-10 bands and 1 hour to assay L32 and GAPDH bands.

Hsp 27 induced almost 7.2 fold increases in mRNA levels, as compared to only adherence stimulated MØ. Hsp 27 induced IL-10 mRNA levels were 3.2 fold higher than the control—MDP+SEB—induced IL-10 mRNA levels, again demonstrating Hsp 27's potency as an IL- 10 inducer. Thus, Hsp 27 induced IL-10 production in MØ is not merely due to an increased rate of translation. Rather, Hsp 27 augments MØ IL-10 production by increasing IL-10 gene transcription and is a more potent stimulus than MDP+SEB.

### Example 4.

### Role of TNFα in Hsp 27 Induction of MØ IL-10 Production

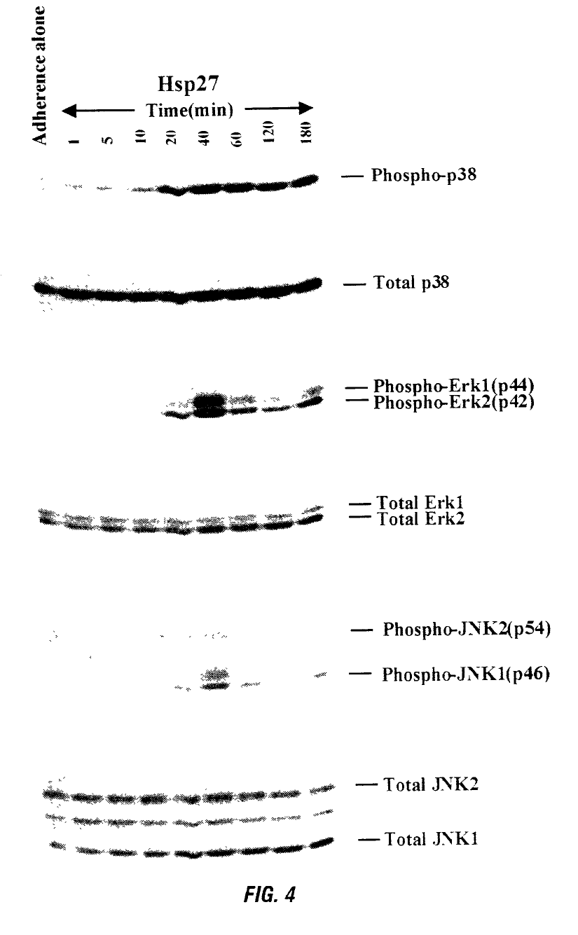

Hsp 60 was known to induce approximately 750 pg/ml TNFα in Mono Mac 6, a human monocyte cell line (7). In addition, TNFα was known to be a potent augmentor of IL-10 production in human MØ (13, 27). TNFα induction occurs prior to IL-10 induction in human MØ after LPS stimulation (31). Thus, exogenously added Hsp 27 could first induce MØ TNFα, which in turn autocrine stimulated the MØ to induce IL-10. A critical requirement for such endogenous induction of TNFα during LPS stimulation of IL-10 in monocytes has been repeatedly reported (27, 32). To test this possibility, we first assessed Hsp 27 induced TNFα production in human MØ. Hsp 27 significantly (p=0.0003) induced TNFα levels in human MØ (FIG. 4A). However, in contrast to Hsp 27's exaggerated MØ IL-10 inducing potential (10 fold vs 3 fold, as compared to MØ IL- 10 inducing potential of SEB+MDP), Hsp 27 and SEB+MDP induced almost identical levels of MØ TNFα (FIG. 4A). In addition, the combination of SEB+MDP+Hsp 27 further significantly (p=0.002) increased MØ TNFα production from 483±74 for Hsp 27 alone to 1737±267 pg/ml These data are in contrast to the failure of the same combination (Hsp+MDP+SEB) to increase MØ IL-10 production over maximal IL-10 production (approximately 3400 pg/ml) induced by Hsp 27 alone.

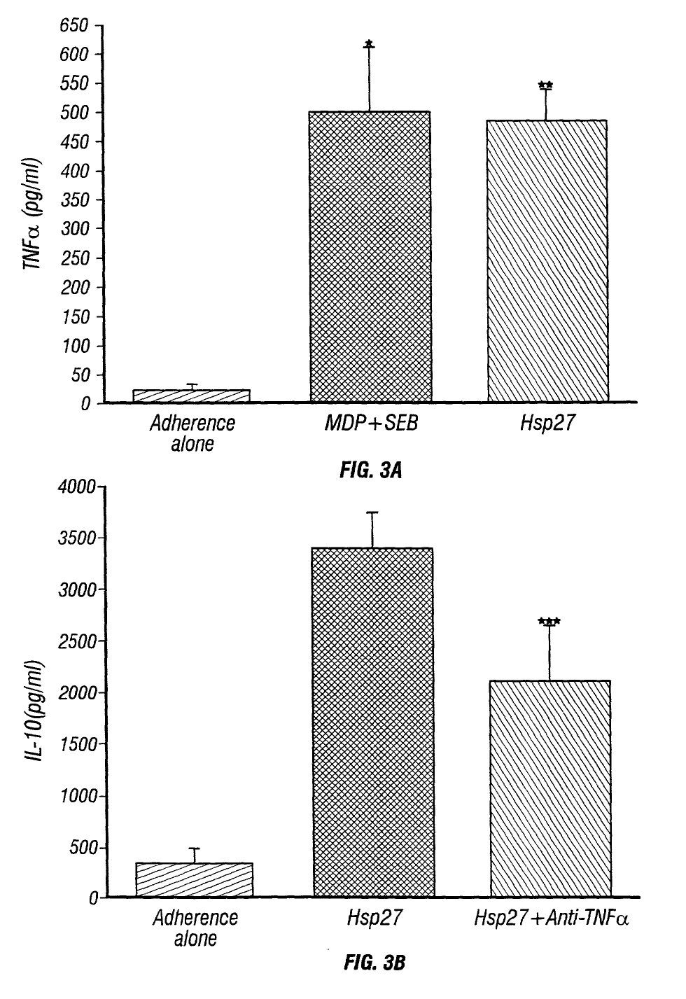

In the next experiments, we added anti-TNFα antibody, along with Hsp 27, to the MØ culture to delineate any critical role of endogenously produced TNFα levels during Hsp 27 induced MØ IL-10 production. As can be seen in FIG. 3B, anti-TNFα antibodies could only partially (approximately 40%) inhibit Hsp 27 induced IL-10 production. In fact, exogenous addition of 100 U/ml TNFα induced only a 1.5 fold increase in IL-10 levels, while addition of Hsp 27 induced an approximately 10 fold increase over adherence stimulated MØ. Therefore, Hsp 27 induced MØ IL-10 production was only partially due to endogenous induction of TNFα and Hsp 27 induced much higher levels of IL-10 compared to its induction of TNFα.

### Example 5.

### Induction of MAPKinase Pathways by Hsp27

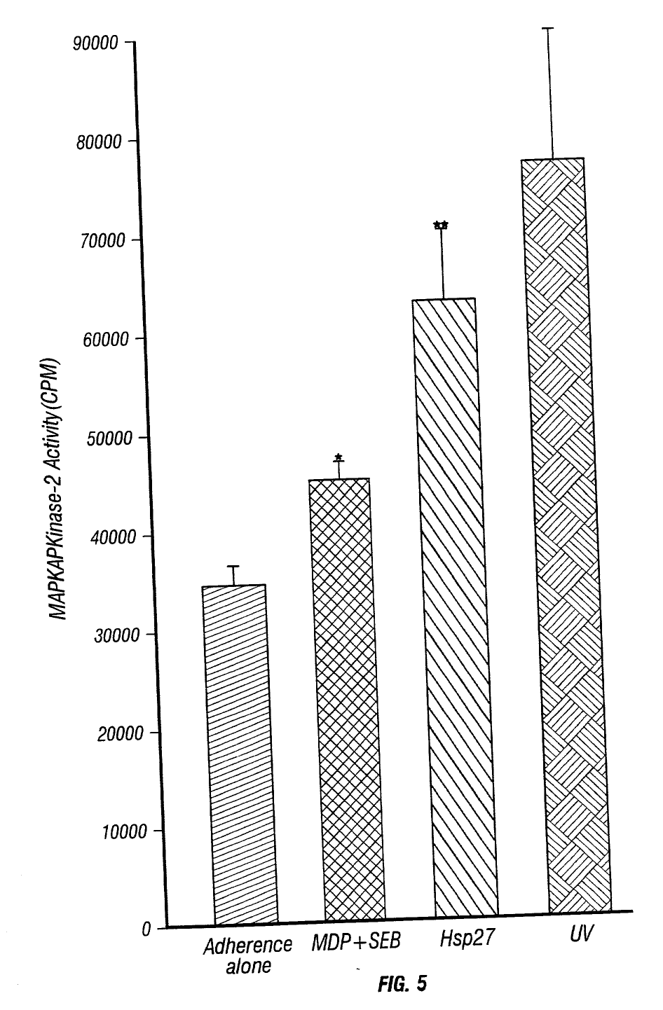

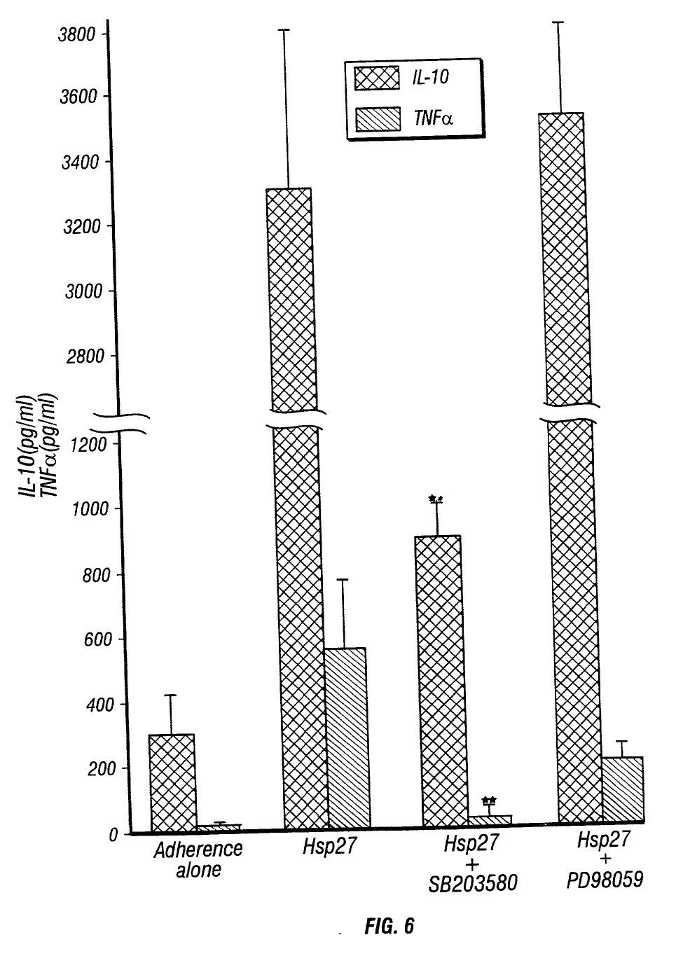

To assess the activation (phosphorylation) of three different mitogen-activated protein kinases (MAPK)—p38, p44/42 (Erk 1/2) and p46/54 (SAPK/JNK-1/2) after Hsp 27 addition, we stimulated human MØ at different time points and measured activated p38, Erk 1/2 and JNK 1/2 using the respective antibodies against the phosphorylated forms of the MAPKs. As can be seen in FIG. 4, Hsp 27 activated all three MAPKs. Phosphorylation of Erk 1/2, JNK 1/2, as well as p38 MAPK was clearly increased at 20 min after addition of Hsp 27. Maximal stimulation was observed at 40 min after Hsp 27 addition for all three MAPKs. However, activation of p38 MAPK persisted up to 180 min when P-p44 Erk, as well as P-p54 and p46 JNK were clearly declining (FIG. 5). In addition, Hsp 27 induction of P-p54 JNK was only minimal compared to its activation of p38 and Erk.

Activation of MAPKAPKinase-2 (a substrate of p38 MAPK) has been shown as necessary to LPS induction of IL-10 in human MØ (13). Therefore, we also assessed the activation of MAPKAPKinase-2 during Hsp 27 induced activation and IL- 10 production of human MØ by in vitro kinase assay, using a sequence of Hsp 27 (KKLNRTSVA; SEQ ID NO: 1) as the substrate. As can be seen in FIG. 5, the immunoprecipitate (using α-MAPKAPKinase-2 sheep polyclonal antibody) from Hsp 27 activated MØ lysate had significantly increased MAPKAPKinase-2 activity versus that from adherence stimulated untreated MØ lysate. The control immunoprecipitate (using sheep IgG) did not have any appreciable MAPKAPKinase-2 activity (data not shown). These data suggest the ability of exogenously added Hsp 27 to activate (phosphorylate) human MØ endogenous Hsp 27. Thus, Hsp 27 is a potent inducer of IL-10 in human MØ but differentially activates the MAPK pathways which play critical roles in inducing monokine production. The next sets of experiments examined which of the different MAPKs had critical roles in Hsp 27 induced IL-10 production by MØ.

### Example 6.

### Role of p38 MAPK in Hsp 27 Induced MØ IL-10 Production.

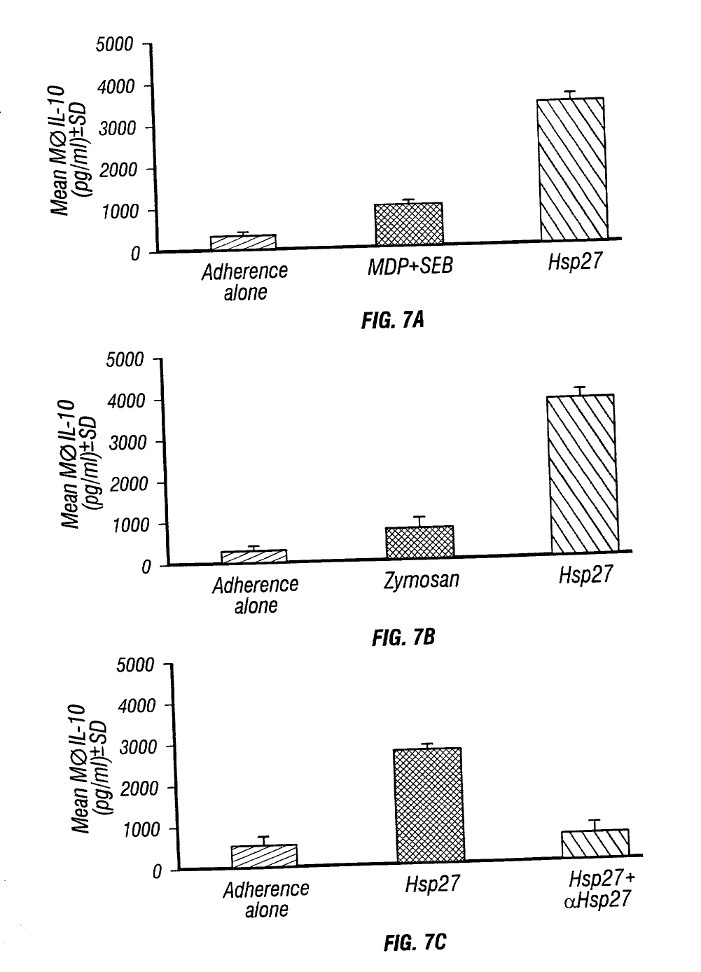

To determine whether there is any essential role of different MAPKinases for induction of MØ IL-10 or TNFα by Hsp 27, we added different MAPKinase inhibitors to the MØ culture before addition of Hsp 27. SB203580 was used to block the effect of p38 MAPKinase, whereas PD98059 was used to inhibit the effect of MEK 1/2 (the enzyme responsible for activation of Erk 1/2) (13, 33). SB203580 could significantly (p=0.002) block Hsp 27 induced IL-10 production (FIG. 7). MØ IL-10 production was inhibited by approximately 80% by SB203580 which also blocked 90% of the TNFα activity induced by Hsp 27, indicating a potential critical role of p38 MAPKinase pathway during induction of both MØ IL-10 and TNFα production by Hsp 27. However, even in the presence of SB203580, Hsp 27 induced a small amount of IL-10, which was still significantly (p=0.002) increased over that of adherence only stimulated MØ (FIG. 7). DMSO control did not have any effect on Hsp 27 induced MØ IL-10 or TNFα production (data not shown). In contrast to the inhibitory effects of SB203580, PD98059 did not have any inhibitory effect on Hsp 27 induced MØ IL-10 production (FIG. 7). The PD98059 was active in these experiments because 68% of the TNFα induced by Hsp 27 was blocked by PD98059 (FIG. 7). These data suggested that activation of the Erk 1/2 pathway is not required for induction of MØ IL-10 by Hsp 27, but that both the Erk 1/2 and p38 pathways are involved in Hsp 27 induction of MØ TNFα.

### Example 7.

### Simultaneous Induction of IL-10 and IL-12 by Hsp27

Large Hsps (Hsp60 and Hsp 70) induce proinflammatory cytokine production by human MØ. Paradoxically, increasing large Hsp levels is beneficial in endotoxin induced systemic inflammatory syndrome, suggesting that Hsps may induce different cytokine responses in unstimulated versus in vivo activated cells. Hsp 27, an essential substrate for a protein kinase in the p38 mitogen activated protein kinase (MAPK) pathway leading to MØ cytokine production, was compared to SEB+MDP for its induction of IL-12, an immunostimulatory cytokine, and of IL-10, an antiinflammatory cytokine, using both normal human MØ and MØ from immunodepressed or immunocompetent trauma patients. Hsp 27 activation requirements for both the MØ Erk and p38 MAPKinase pathways were evaluated by Western blot for P-Erk and P-p38, by kinase assay of MAPKAPK-2, and with the specific MAPK inhibitors SB203580 (p38) or PB98059 (Erk). Hsp 27 stimulated normals' or immunocompetent trauma patient's MØ to 2.5-3.5 greater increases in Il-10 and IL-12 than SEB+MDP.

Even trauma patients with depressed MØ IL-10 levels responded to Hsp 27 with a 2.5 fold increase in IL-10 production as compared to induction with SEB+MDP. In striking contrast, patient MØ with highly depressed IL-12 production to SEB+MDP produced 8-10 fold more IL-12 in response to Hsp 27. Additionally, although Hsp 27-induced MØ IL-10 critically depends on p38 MAPK pathway activation, its induction of IL -12 depends neither on the p38 nor the ERK 1/2 pathways. This suggests that Hsp 27 is not only a potent stimulus of both IL-10 and IL-12, but its potency for IL-12 induction differs depending on MØ activation status.

Hsp 27 is not only a potent stimulus for induction of IL-10; it is also a potent simultaneous inducer of IL-12 in human monocytes, as compared to other stimulants such as a combination of SEB and MDP (Table 1).

TABLE 1
Simultaneous induction of IL-10 and IL-12 by Hsp 27 in
Normal control and trauma patients' monocytes

| | IL-10(pg/10⁶ cells/ml) | | | IL-12(pg/10⁶ cells/ml) | | |
|---|---|---|---|---|---|---|
| | x ± SEM | | | x ± SEM | | |
| | Unstim. | MDP^{c} + SEB | HSP 27^{d} | Unstim. | MDP + SEB | Hsp 27 |
| Normal^{a} | 617 ± 124 | 1708 ± 238 | 5267 ± 610 | 213 ± 72 | 1255 ± 285 | 3428 ± 1102 |
| Patient^{b} | 98 ± 30 | 268 ± 80 | 670 ± 195 | 22 ± 8 | 96 ± 38 | 598 ± 140 |

^{a}Normal □ n = 14
^{b}Patient □ n = 14
^{c}MDP □ 20 μg/ml
SEB □ 0.5 μg/ml
^{d}Hsp 27 □ 2 μg/ml
^{c} & ^{d}□ moncytes were stimulated for 16-18 hours with these stimulants.

Both IL-10 (anti-inflammatory) and IL-12 (immunostimulatory) are significantly (p<0.001) depressed in trauma patients who have high multiple organ dysfunction syndrome (MODS) scores. Therefore, the ability of Hsp 27 to induce IL-10 and IL-12 in monocytes from immunosuppressed patients was assessed. HSP 27 induced an approximately 3 fold increase in both IL1- and IL-12, as compared to SEB+MDP, in normal human monocytes. Similar to normal moncyte data, Hsp 27 induced an approximately 2.5 fold increase in IL-10 production (as compared to induction with SEB+MDP) in the monocytes of patients. More surprisingly Hsp 27 could simultaneously induce a greater than 6 fold increase in IL-12 production (as compared to SEB+MDP) in patient's monocytes (Table 1).

This example also demonstrates methods of evaluating the efficacy of Hsp 27 treatment in patients. Efficacy can also be assessed by reduction or elimination of patient symptoms either by patient report or by other suitable means of evaluating the patient's physical condition including laboratory tests, evaluation of synovial fluid (for example in rheumatoid arthritis), and radiographic methods.

### Example 8

### Animal Model for Testing the Anti-inflammatory Effects of Hsp 25

Studies were performed to analyze the adverse effect of Hsp 25 (a murine analogue of Hsp27 ) in rats. 10 μg of Hsp25 did not induce any adverse effects in normal rats (˜250 gm body weight). To examine the effects in an animal model, the cecal ligation and puncture model (CLP) in rats can be used. (Chaudry, et al, 1993, In: Schlag, G. and Redl, H. (Eds), Pathophysiology of shock, Sepsis, and Organ Failure. Springer-Verlag, pp. 1048-1059.) CLP induces sepsis in rats. Hsp25 is administered to the CLP animals and the anti-inflammatory effect of Hsp25 in CLP rats is evaluated. The effectiveness of Hsp25 in simultaneously inducing IL-10 and IL-12 in human monocytes in the rat model, is reasonably predictive of the efficacy of Hsp27 as an anti-inflammatory agent in humans.

### Example 9

### Further Studies on Induction of IL-10 and IL-12 Production by Human MØ

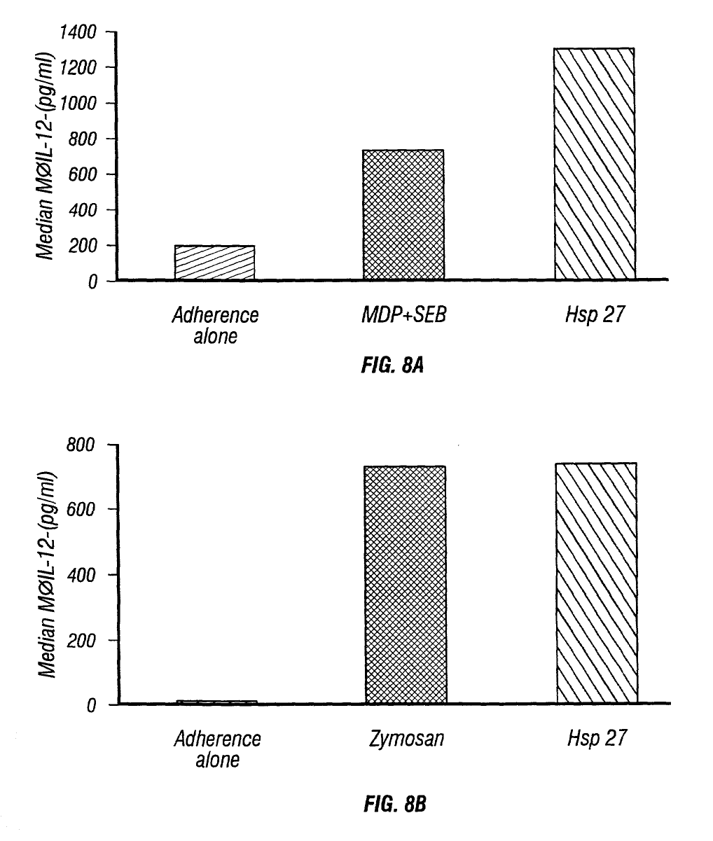

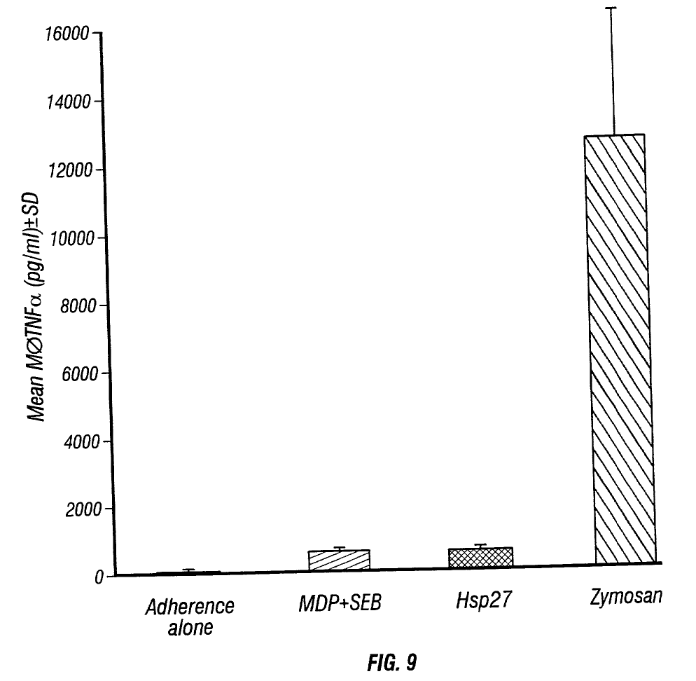

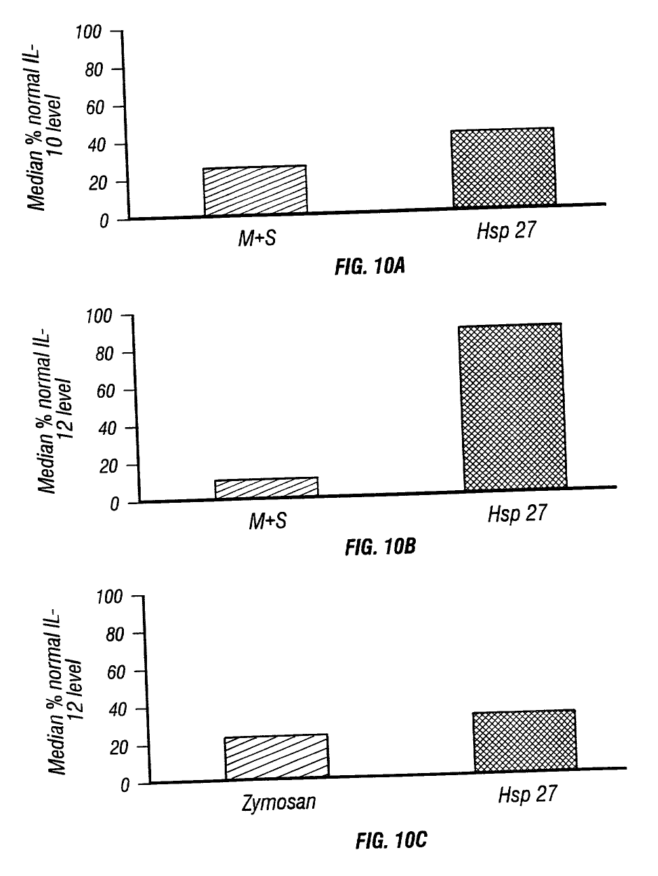

The following tests were conducted to assess the ability of Hsp27 to restore depressed monocyte IL-10 and IL-12 levels in human trauma patients. Blood was collected (35 ml) from patients suffering mechanical trauma (injury severity score >18) or thermal trauma (3rd degree burns over at least 30% of body surface area). Peripheral broad mononuclear cells (PBMC) were isolated using Ficol density gradient centrifugation. MØ were isolated by depletion of T cells and B cells with anti-CD2 and anti-CD19 coated magnetic beads. The isolated MØ were subjected to one of the following: (a) 20 μg/ml MDP plus 0.5 μg/ml SEB, (b) 2 μg/ml Hsp27, or (c) 50 μg/ml Zymosan A. IL-10 and IL-12 levels in cell culture supernates were assessed by conventional ELISA techniques. TNFα was also assayed by ELISA. Data from these experiments are summarized in FIGS. 7-9.

### Example 10

### Promotion of Dendritic Cell Maturation by Hsp27

We assessed the effect of exogenous Hsp27 on MØ conversion (differentiation) into immature dendritic cells. We also assessed the effect of exogenous Hsp27 on maturation of dendritic cells into fully capable antigen-presenting cells.

Hsp27 added to MØ cultures at the initiation of conversion inhibited differentiation of MØ to dendritic cells mediated or promoted by the combination of IL-4 plus GM-CSF. In contrast, Hsp27 added to the MØ cultures after initial differentiation into DC (day 4 of induction) strongly promoted maturation of immature DC (CD14⁻, CD1a⁺) to highly potent, mature, antigen-presenting DC (CD14⁻, CD1a^{±}, CD83⁺). These more mature dendritic cells displayed increased maturation markers. More significantly, these mature dendritic cells displayed an increase ability to activate T lymphocyte proliferation in the mixed lymphocyte response (MLR). Data on CD83 expression and MLR proliferation are summarized in Table 2 below.

TABLE 2

| | CD83 Expressions | MLR Proliferation |
|---|---|---|
| IL-4⁺ GM-CSF | 12% positive cells | 8 × 10⁺ |
| IL-4⁺ GM-CSF + HSP-27 | 89.5% | 13 × 8 × 10⁺ |

Our data suggested that in addition to regulating the inflammatory host status, Hsp27 treatment may also simultaneously increase T cell activation, thereby reducing the T cell dysfunction that occurs in severe inflammatory diseases.

### REFERENCES CITED

1. Feldmann, M., Brennan, F. M., and Maini, R. 1998. Cytokines in autoimmune disorders. Int. Reviews. of Immunol 17:217.

2. Bahrami, S., Redl, H., Yao, Y. M., and Schlag, G. 1996. Involvement of bacteria/endotoxin translocation in the development of multiple organ failure. Curr. Top. Microbiol. Immunol. 216:239.

3. Jindal, S. 1996. Heat shock proteins: applications in health and disease. Trends in Biotech. 14:17.

4. Fanning, N. F. and Redmond, H. P. 1998. Heat shock proteins in the regulation of the apoptotic response. Sepsis 2:47.

5. Mizzen, L. 1998. Immune responses to stress proteins: Applications to infectious disease and cancer. Biotherapy 10:173.

6. Van Eden, W., van der Zee, R., Paul, A. G. A., Prakken, B. J., Wendling, U., Anderton, S. M., and Wauben, M. H. M. 1998. Do heat shock proteins control the balance of T-cell regulation in inflammatory diseases? Elsevier Sci. Ltd. 19:303.

7. Chen, W., Syldath, U., Bellmann, K., Burkart, V., and Kolb, H. 1999. Human 60-kDa heat-shock protein: A danger signal to the innate immune system. J. Immunol. 162:3212.

8. Colaco, C. A. L. S. 1998. Towards a unified theory of immunity: Dendritic cells, stress proteins and antigen capture. Cell. Mol. Biol. 44:883.

9. Van Roon, J. A. G., Van Eden, W., Van Roy, J. L. A. M., Lafeber, F. J. P. G., and Bijlsma, J. W. J. 1997. Stimulation of suppressive T cell responses by human but not bacterial 60-kD heat-shock protein in synovial fluid of patients with rheumatoid arthritis. J. Clin. Invest. 100:459.

10. Gaston, J. S. H. 1998. Heat shock proteins as potential targets in the therapy of inflammatory arthritis. Biotherapy 10:197.

11. Multhoff, G. and Botzler, C. 1998. Heat-shock proteins and the immune response. Annals of the NY Academy of Sciences 851:86.

12. Beech, J. T., Siew, L. K., Ghoraishian, M., Stasiuk, L. M., Elson, C. J., and Thompson, S. J. 1997. CD4+Th2 cells specific for mycobacterial 65-kilodalton heat shock protein protect against pristane-induced arthritis. J. Immunol. 159:3692.

13. Foey, A. D., Parry, S. L., Williams, L. M., Feldmann, M., Foxwell, B. M. J., and Brennan, F. M. 1998. Regulation of monocyte IL-10 synthesis by endogenous IL-1 and TNF-α: Role of the p38 and p42/44 mitogen-activated protein kinases. J. Immunol. 160:920.

14. Joyce, D. A., Gibbons, D. P., Green, P., Steer, J. H., Feldmann, M., and Brennan, F. M. 1994. Two inhibitors of pro-inflammatory cytokine release, interleukin-10 and interleukin-4, have contrasting effects on release of soluble p75 tumor necrosis factor receptor by cultured monocytes. Eur. J. Immunol. 24:2699.

15. Niiro, H., Otsuka, T., Kuga, S., Nemoto, Y., Abe, M., Hara, N., Nakano, T., Ogo, T., and Niho, Y. 1994. IL-10 inhibits prostaglandin E2 production by lipopolysaccharide-stimulated monocytes. Int. Immunol. 6:661.

16. Ciocca, D. R., Oesterreich, S., Chamness, G. C., McGuire, W. L., and Fuqua, A. W. 1993. Biological and clinical implications of heat shock protein 27000 (Hsp27): a review. J. Nat'l. Canc. Inst. 85:1558.

17. Sutherland, C. L., Krebs, D. L., and Gold, M. R. 1999. An 11-amino acid sequence in the cytoplasmic domain of CD40 is sufficient for activation of c-Jun N-terminal kinase, activation of MAPKAP kinase-2, phosphorylation of IεBα, and protection of WEHI-231 cells from anti-IgM-induced growth arrest. J. Immunol. 162:4720.

18. Gazzinelli, R. T., Oswald, I. P., James, S. L., and Sher, A. 1992. IL-10 inhibits parasite killing and nitrogen oxide production by IFN-γ activated macrophages. J. Immunol. 148:1792.

19. Roilides, E., Dimitriadou, A., Kadiltsoglou, I., Sein, T., Karpouzas, J., Pizzo, P. A., and Walsh, T. J. 1997. IL-10 exerts suppressive and enhancing effects on antifungal activity of mononuclear phagocytes against Aspergillus fumigatus. J Immunol. 158:322.

20. Maizels, E. T., Peters, C. A., Kline, M., Cutler, R. E.,Jr., Shanmugam, M., and Hunzicker-Dunn, M. 1998. Heat-shock protein-25/27 phosphorylation by the delta isoform of protein kinase C. Biochem. J 332:703.

21. Ahlers, A., Belka, C., Gaestel, M., Lamping, N., Sott, C., Herrmann, F., and Brach, M. A. 1994. Interleukin-1-induced intracellular signaling pathways converge in the activation of mitogen-activated protein kinase and mitogen-activated protein kinase-activated protein kinase 2 and the subsequent phosphorylation of the 27-kilodalton heat shock protein in monocytic cells. Mol. Pharmacol. 46:1077.

22. Sanghera, J. S., Weinstein, S. L., Aluwalia, M., Girn, J., and Pelech, S. L. 1996. Activation of multiple proline-directed kinases by bacterial lipopolysaccharide in murine macrophages. J. Immunol. 156:4457.

23. Fanelli, M. A., Carrion, F. D. C., Dekker, J., Shoemaker, J., and Ciocca, D. R. 1998. Serological detection of heat shock protein hsp27 in normal and breast cancer patients. Can Epidem., Biomarkers & Prevention 7:791.

24. Conroy, S. E., Sasieni, P. D., Amin, V., Wang, D. Y., Smith, P., Fentiman, I. S., and Latchman, D. S. 1998. Antibodies to heat-shock protein 27 are associated with improved survival in patients with breast cancer. British J. of Cancer 77:1875.

25. Piotrowicz, R. S., Hickey, E., and Levin, E. G. 1998. Heat shock protein 27 kDa expression and phosphorylation regulates endothelial cell migration. FASEB J. 12:1481.

26. Creery, D. and Kumar, A. 1998. The potential for interleukin-10-directed therapies in human sepsis. Sepsis 2:209.

27. Platzer, C., Meisel, C., Vogt, K., Platzer, M., and Volk, H.-D. 1995. Up-regulation of monocytic IL-10 by tumor necrosis factor-α and cAMP elevating drugs. Int. Immunol. 7:517.

28. Miller-Graziano, C. L., De, A. K., and Kodys, K. 1995. Altered IL-10 levels in trauma patients' MØ and T lymphocytes. J. Clin. Immunol 15:93.

29. Schwenger, P., Bellosta, P., Vietor, I., Basilico, C., Skolnik, E.Y., and Vilcek, J. 1997. Sodium salicylate induces apoptosis via p38 mitogen-activated protein kinase but inhibits tumor necrosis factor-induced c-Jun N-terminal kinase stress-activated protein kinase activation. Proc. Natl. Acad. Sci. USA 94:2869.

30. Bondeson, J., Browne, K. A., Brennan, F. M., Foxwell, B. M. J., and Feldmann, M. 1999. Selective regulation of cytokine induction by adenoviral gene transfer of IεBα into human macrophages: Lipopolysaccharide-induced, but not zymosan-induced, proinflammatory cytokines are inhibited, but IL-10 is nuclear factor-εB independent. J. Immunol. 162:2939.

31. Donnelly, R. P., Freeman, S. L., and Hayes, M. P. 1995. Inhibition of IL-10 expression by IFN-γ up-regulates transcription of TNF-α in human monocytes. J. Immunol. 155:1420.

32. Wanidworanun, C. and Strober, W. 1993. Predominant Role of Tumor Necrosis Factor-α in Human Monocyte IL-10 Synthesis. J. Immunol. 151:6853.

33. Scherle, P. A., Jones, E. A., Favata, M. F., Daulerio, A. J., Covington, M. B., Nurnberg, S. A., Magolda, R. L., and Trzaskos, J. M. 1998. Inhibition of MAP kinase kinase prevents cytokine and prostaglandin E₂ production in lipopolysaccharide-stimulated monocytes. J. Immunol. 161:5681.

34. Guay, J., Lambert, H., Gingras-Breton, G., Lavoie, J. N., Huot, J., and Landry, J. 1997. Regulation of actin filament dynamics by p38 map kinase-mediated phosphorylation of heat shock protein 27. J. Cell Sci. 110:357.

35. Srivastava, P. K., Menoret, A., Basu, S., Binder, R. J., and McQuade, K. L. 1998. Heat shock proteins come of age: Primitive functions acquire new roles in an adaptive world. Immunity 8:657.

36. Al-Shamma, M. R. R., McSharry, C., McLeod, K., McCruden, E. A. B., and Stack, B. H. R. 1997. Role of heat shock proteins in the pathogenesis of cystic fibrosis arthritis. Thorax 52:1056.

37. Tezel, G., Seigel, G. M., and Wax, M. B. 1998. Autoantibodies to small heat shock proteins in glaucoma. Invest. Opthal. & Visual Sci. 39:2277.

38. Knauf, U., Jakob, U., Engel, K., Buchner, J., and Gaestel, M. 1994. Stress- and mitogen-induced phosphorylation of the small heat shock protein Hsp25 by MAPKAP kinase 2 is not essential for chaperone properties and cellular thermoresistance. The EMBO Journal 13:54.

39. Mehlen, P., Mehlen, A., Guillet, D., Preville, X., and Arrigo, A-P. 1995. Tumor necrosis factor-α induces changes in the phosphorylation, cellular localization, and oligomerization of human hsp27, a stress protein that confers cellular resistance to this cytokine. J. of Cell. Biochem. 58:248.

40. Beresford, P. J., Madhuri, J., Friedman, R. S., Yoon, M. J., and Lieberman, J. 1998. A role for heat shock protein 27 in CTL-mediated cell death. J. Immunol. 161:161.

41. LeMoine, O., Stordeur, P., Schandene, L., Marchant, A., deGroote, D., Goldman, M., and Deviere, J. 1996. Adenosine enhances IL-10 secretion by human monocytes. J. Immunol. 156:4408.

42. Liu, L. M., Rich, B. E., Inobe, J., Chen, W. J., and Weiner, H. L. 1997. A potential pathway of Th2 development during primary immune response—IL-10 pretreated dendritic cells can prime naive CD4⁺ T cells to secrete IL-4. Adv. Exp. Med. Biol. 417:375.

43. Skeen, M. J., Miller, M. A., Shinnick, T. M., and Ziegler, H. K. 1996. Regulation of murine macrophage IL-12 production. J. Immunol. 156:1196.

44. Matzinger, P. 1998. An innate sense of danger. Seminars in Immunology 10:399.

45. Dong, C., Yang, D. D., Wysk, M., Whitmarsh, A. J., Davis, R. J., and Flavell, R. A. 1998. Defective T cell differentiation in the absence of Jnk 1. Science 282:2092.

46. Lalla, E., Lamster, I. B., Feit, M., Huang, L., and Schmidt, A. M. 1998. A murine model of accelerated periodontal disease in diabetes. J. of Periodontal Res. 33:387.

47. Ablamunits, V., Elias, D., Reshef, T., and Cohen, I. R. 1998. Islet T cells secreting IFN-γ in NOD mouse diabetes: Arrest by p277 peptide treatment. J. Autoimmunity 11:73.

48. Lander, H. M., Tauras, J. M., Ogiste, J. S., Hori, O., Moss, R. A., and Schmidt, A. M. 1997. Activation of the receptor for advanced glycation end products triggers a p21 (ras)-dependent mitogen-activated protein kinase pathway regulated by oxidant stress. J. Biol. Chem. 272:17810.

### OTHER EMBODIMENTS

Other embodiments are within the following claims.

What is claimed is:

1. A method of inhibiting an inflammatory response in a mammal, the method comprising administering to the mammal a therapeutically effective amount of Hsp 27.

2. A method of inducing IL-10 production in a mammal, the method comprising administering to the mammal an effective amount of Hsp 27.

3. A method of inducing IL-12 production in a mammal, the method comprising administering to the mammal an effective amount of Hsp 27.

4. A method of simultaneously inducing IL-10 production and IL-12 production in a mammal, the method comprising administering to the mammal an effective amount of Hsp 27.

5. The method of claim 1, wherein the therapeutically effective amount is 1 μg/kg to 160 μg/kg.

6. The method of claim 5, wherein the therapeutically effective amount is 2 μg/kg to 80 μg/kg.

7. The method of claim 6, wherein the therapeutically effective amount is 4 μg/kg to 40 μg/kg.

8. An anti-inflammatory composition comprising an effective amount of Hsp 27 and a pharmaceutically acceptable carrier.

9. A method of promoting dendritic cell maturation, the method comprising:
isolating monocytes from blood without triggering activation;
culturing the monocytes ex vivo;
inducing conversion of the monocytes into immature dendritic cells; and
contacting the dendritic cells with an effective amount of Hsp27 for an effective length of time,
thereby promoting maturation of the dendritic cells.

10. The method of claim 9, wherein inducing conversion of the monocytes into immature dendritic cells comprises culturing the monocytes in a medium comprising IL-4 and GMCSF for an effective conversion time.

11. The method of claim 10, wherein the effective conversion time is 2 to 5 days.

12. The method of claim 9, wherein the effective amount of Hsp27 is 0.1 μg/ml to 500 μg/ml.

13. The method of claim 9, wherein the effective amount of Hsp27 is 1 μg to 100 μg.

14. The method of claim 9, wherein the effective amount of Hsp27 is 5 μg to 50 μg.

15. A method of enhancing an immune system response in a human patient, the method comprising:
collecting a sample of blood from the patient;
isolating monocytes from the blood without triggering activation of the monocytes;
culturing the monocytes ex vivo;
inducing conversion of the monocytes into immature dendritic cells;
promoting maturation of the dendritic cells by contacting the dendritic cells with an effective amount of Hsp27 for an effective length of time; and
reintroducing the dendritic cells into the patient.

16. The method of claim 15, further comprising the step of contacting the dendritic cells with an antigen after promoting maturation of the dendritic cells, and before reintroducing the dendritic cells into the patient.

17. The method of claim 16, wherein the antigen is selected from the group consisting of a human tumor antigen, a bacterial antigen, and a viral antigen.
